(19) United States
(12) Patent Application Publication
(10) Pub. No.: US 2005/0181040 A1
(43) Pub. Date: Aug. 18, 2005

(54) Progestagenic dosage unit

(76) Inventor: Pieter De Haan, Bh Oss (NL)

Correspondence Address: INTERVET U.S.;PATENT DEPARTMENT, PO BOX 318, MILLSBORO, DE, 19966-0318, US

(21) Appl. No.: 10/515,670

(22) PCT Filed: May 22, 2003
(86) PCT No.: PCT/EP03/50189

(30) Foreign Application Priority Data
May 29, 2002 (EP) 02077097.0

(51) Int. Cl. A61K 9/48; A61K 31/56

(52) U.S. Cl. 424/451; 514/169; 264/109

(57) ABSTRACT

The subject invention concerns a method of producing pharmaceutical dosage units for oral administration comprising a progestagenic compound which perorally exerts progestagenic activity equivalent to that of the progestagen desogestrel.

### FIELD OF THE INVENTION

This invention pertains to the field of (male and female) contraception, (male and female hormone replacement therapy (HRT) and the treatment and prevention of gynecological disorders.

### BACKGROUND

The progestogen desogestrel (13-ethyl-11-methylene-18,19-dinor-17à-pregn-4-en-20-yn-17-ol) is an active substance used commonly in dosage units for contraception and HRT which are marketed in several countries in different compositions, either with or without ethinyl estradiol.

Desogestrel however, has the unwanted property of transferring from dosage units into the surrounding local environment. As a result thereof, the quantity of desogestrel contained within the dosage unit may drop below the stated level within a relatively short period of time. This undesired physical property of desogestrel is of particular concern for stability when the dosage unit comprises very low dosages of desogestrel.

Desogestrel in tablets also has a moderate chemical stability, i.e. it decreases in concentration during storage and unwanted levels of decomposition products are formed.

Various methods for preparing dosage units containing desogestrel have been disclosed: EP 503521 discloses a method of making dosage units comprising low-dosed potent steroids such as desogestrel by dry-mixing the steroid with judiciously selected excipients, viz. spray-dried polyalcohol or granulated α-lactose monohydrate. EP 657161 discloses a method of preparing granules and dosage units comprising steroids such as desogestrel, wherein the desogestrel is dissolved in an organic solvent together with a lubricant, and wherein the resulting solution is distributed over a carrier. EP 659432 discloses a method of producing sugar-coated dosage units in which steroids such as desogestrel are more stably contained. EP 927556 discloses different coatings for the same purpose. EP 688565 discloses oral dosage units comprising desogestrel wherein the desogestrel is solved or dispersed in a matrix comprising a lipid, oil, or wax. EP 78249 describes a process of making dosage units containing desogestrel by wet granulation. EP 707848 describes solid pharmaceutical compositions comprising a steroid such as desogestrel and an excipient capable of binding water. EP 833642 discloses compressed dry-granulation desogestrel tablets.

It has now surprisingly been found that etonogestrel can be used to achieve a pharmaceutical improvement in stability over desogestrel in oral dosage units. This active ingredient of the subject dosage units prepared by solvent-granulation does not migrate into the surrounding local environment thereby solving the problem of dosage units having lower content than stated. Moreover, the subject dosage units made by the subject process have an optimal combination of content uniformity and homogeneity. The subject process further avoids complexity and is highly reproducible. Most of all, the dosage units of the subject invention are much less prone to decomposition and can thus be stored over long periods of time.

Even though some of the above discussed references already mention 3-keto-desogestrel (etonogestrel), the active metabolite of desogestrel, as an alternative to using desogestrel, none of them actually disclose the production of tablets (of any kind) with etonogestrel nor do any of these references show the excellent stability profile of such tablets.

### SUMMARY OF THE INVENTION

The subject invention provides a method of producing pharmaceutical dosage units for oral administration comprising a progestagenic compound which perorally exerts progestagenic activity equivalent to that of the progestagen desogestrel, the method comprising (i) dissolving the progestagenic compound in an organic solvent to form a solution, (ii) mixing the solution comprising the progestogenic compound with a carrier, (iii) optionally granulating the resulting mixture, (iv) drying the mixture, (v) admixing the mixture with excipients and (vi) turning the resulting mixture into dosage units, characterized in that the progestagenic compound is etonogestrel.

### DETAILED DESCRIPTION

The progestagen etonogestrel is also known as 3-ketodesogestrel. Its chemical name is (17α) 13-ethyl-17-hydroxy-11-methylene-18,19-dinorpregn-4-en-20-yn-3-one (also 11,11-methylene-17α-ethynyl-17β-hydroxy-18-methyl-Δ⁴-estren-3-one) and it can be prepared as disclosed in e.g. U.S. Pat. No. 3,927,046.

The subject invention provides a method of producing pharmaceutical dosage units for oral administration comprising a progestagenic compound which perorally exerts progestagenic activity equivalent to that of the progestagen desogestrel, the method comprising (i) dissolving the progestagenic compound in an organic solvent to form a solution, (ii) mixing the solution comprising the progestogenic compound with a carrier, (iii) optionally granulating the resulting mixture, (iv) drying the mixture, (v) admixing the mixture with excipients and (vi) turning the resulting mixture into dosage units, characterized in that the progestagenic compound is etonogestrel. In one embodiment, the mixture is filled into a capsule so as to make a dosage unit in the form of a capsule. In another embodiment, the mixture is tabletted so as to make a dosage unit in the form of a tablet.

The subject invention further contemplates a use of etonogestrel for the manufacture of a progestagenic tablet for oral administration, wherein the tablet is obtainable by a process comprising (i) dissolving the progestagenic compound in an organic solvent to form a solution, (ii) mixing the solution comprising the progestogenic compound with a carrier, (iii) optionally granulating the resulting mixture, (iv) drying the mixture, (v) admixing the mixture with excipients and (vi) tabletting the resulting mixture thereby obtaining the progestogenic tablet.

As used herein, the term “dosage unit” generally refers to physically discrete units suitable as unitary dosages for humans or animals, each containing a predetermined quantity of active material calculated to produce the desired effect.

Methods and compositions for making such dosage units are well-known to those skilled in the art. For example, methods and compositions for making tablets, capsules and pills containing active ingredients, are described in the standard reference, Chase et al., Remington's Pharmaceutical Sciences, (16th ed., Mack Publishing Co., Easton. Pa., U.S.A., 1980) (“Remington's”), at pages 1553 through 1584. Methods of making powders, and their composition are described at pages 1535 through 1552 of the reference. Methods of coating pharmaceutical dosage forms are described at pages 1585 to 1593 of Remington's. The contents of these pages are hereby incorporated by this reference.

For making dosage units, e.g. tablets, the use of conventional additives, e.g. fillers, glidants, flow enhancers, colorants, polymeric binders, lubricants and the like is contemplated. In general any pharmaceutically acceptable additive which does not interfere with the function of the active compound can be used in the subject invention.

Suitable carriers with which the compositions can be administered include lactose, starch, cellulose derivatives, calcium phosphates and granulates made thereof and the like used in suitable amounts. Lactose is a preferred carrier. Mixtures of carriers (sometimes in the form of pharmaceutical granulates) can also be used.

A process of manufacturing the tablets of the invention comprises mixing predetermined quantities of 3-keto-desogestrel with predetermined quantities of excipients and converting the dried and homogeneous mixture into dosage units containing preferably 10 to 300 μg 3-keto-desogestrel. Converting the mixture into dosage units generally involves compressing the mixture into a tablet or filling a capsule with a dried mixture.

A preferred process of manufacturing the pharmaceutical product according to the subject invention involves incorporating the desired dosages of 3-keto-desogestrel into a tablet by known techniques.

The dose of etonogestrel used in the subject invention lies in the range of 10-300 μg. A preferred range of the dose of etonogestrel is 15-250 μg. Another preferred range is 15-150 μg. Yet another preferred range is 35-150 μg and another preferred range is 70-80 μg.

The process of the invention can generally be carried out as follows:

Etonogestrel is dissolved in a suitable solvent (organic solvent, a mixture of organic solvents, a mixture of organic solvent(s) and water). The solution is distributed in mixing equipment over the carrier. After distribution the resulting mixture is dried under continued mixing. The dried mixture can be screened when required and subsequently be admixed with lubricants and glidants. The final admixture is filled into capsules or compressed into tablets. The tablets can be provided with a film-coat or sugar-coat.

The subject invention also contemplates that the pharmaceutical dosage units of the subject invention may further comprise an estrogen, an anti-progestin or an androgen.

The present invention is further described in the following examples which are not in any way intended to limit the scope of the invention as claimed.

### EXAMPLES

### Example 1

The active ingredients were processed to a homogenous granulate comprising per tablet:

| | | |
|---|---|---|
| desogestrel | 150 | μg |
| EE | 30 | μg |
| dl-alpha-tocopherol | 0.080 | mg |
| potato starch | 10.10 | mg |
| amylopectine | 1.77 | mg |
| magnesium stearate | 0.50 | mg |
| lactose | to 100.0 | mg |

A batch (60000 tablets) was manufactured as follows: to a basic granulate (5955 grams) comprising potato starch, amylopectine, and lactose, a solution of desogestrel (9.000 grams), ethinylestradiol (EE) (1.800 grams) and dl-alpha-tocopherol (4.800 grams) in acetone (5241 grams) was added and mixed. After evaporation of the acetone the final granulate was admixed with magnesium stearate, resulting in a homogeneous active granulation. The admixed granulation was compressed to flat-faced, 6 mm round tablets of 100 mg.

### Example 2

The active ingredients were processed to a homogenous granulate comprising per tablet:

| etonogestrel | 15 | μg |
|---|---|---|
| ethinyl estradiol | — | |
| dl-alpha-tocopherol | 0.050 | mg |
| potato starch | 5.0 | mg |
| amylopectin | 0.9 | mg |
| magnesium stearate | 0.250 | mg |
| lactose | to 50 | mg |

A batch of 20000 tablets was manufactured by mixing a solution of the active ingredients in acetone with the dry basic granulate. After drying and mixing the granulate under vacuum, the resulting granulate was admixed with magnesium stearate.

The admixed granulate was compressed to tablets weighing 50 mg, with a diameter of 4.5 mm using round flat-faced punches with beveled edges.

### Example 3

The tablets of Examples 1 and 2 were submitted to storage at 25° C. and ambient relative humidity. The content of the tablets (expressed in % of the initial content) and the % of decomposition products formed is given below:

| | | Decomposition product |
|---|---|---|
| | Content after storage | after storage |
| Etonogestrel tablets | | |
| (15 ug in 50 mg) | | |
| 6 months | 99.7% | <0.5% |
| Desogestrel tablets | | |
| (150 ug in 100 mg) | | |
| 6 months | 97.5% | 3.5% |
| 12 months | 93.5% | 7% |

The tablets of Example 2, containing etonogestrel, are clearly much more stable during storage than the tablets of Example 1, containing desogestrel.

One would have expected the opposite to be the case: one would have expected that the higher the concentration of the progestogenic active ingredient, the higher the chemical stability.

Surprisingly however it was found that the (50 mg) tablets of Example 2 containing only 15 μg etonogestrel were much more stable during storage than the (100 mg) tablets of Example 1 containing a concentration of five times as much progestogen, i.e. 150 μg desogestrel.

### Example 4

The Gral 10 high speed granulator is filled with 992.6 g of basic granulate. After dissolving the 3-ketodesogestrel (2.36 g) in 100 ml of ethanol in a beaker, the solution is added to the granulate mass. In addition, 25 ml of ethanol is used to rinse the beaker. The resulting wetted granulate is mixed for 2.5 minutes at mixer position 430 rpm and granulator position 1. After removing the granulate of the chopper and breaker of the Gral 1, mixing is continued for 2.5 minutes. The mass is dried in a vacuum cabinet for 4 hours under diminished pressure at 40° C. The dried mass is screened through a 710 μm sieve. The resulting mass is admixed with magnesium stearate in the Gral high speed mixer (mixer 110 rpm, granulator position 0). Tablets of 65 mg with a diameter of 5 mm and a radius of convexity of 7.5 mm have been compressed from the admixture using a Korsch PH106 rotary press.

The tablets have been provided with a film-coat using the Glatt-labcoater.

Composition of the Film-Coated Tablets

| | | |
|---|---|---|
| etonogestrel | 150 | μg |
| magnesium stearate | 0.325 | mg |
| basic granulate to | 65 | mg |
| Coating layer: | | |
| Hydroxypropylmethylcellulose E15 | 0.75 | mg |
| Talc | 0.1875 | mg |
| Polyethylene glycol 400 | 0.15 | mg |
| Titanium dioxide | 0.1125 | mg |
| Composition of the basic granulate: | | |

| | |
|---|---|
| lactose | 87% |
| corn starch | 10% |
| polyvinylpyrrolidone(PVP) | 3% |

1. A method of producing pharmaceutical dosage units for oral administration comprising a progestagenic compound which perorally exerts progestagenic activity equivalent to that of the progestagen desogestrel, comprising:
(i) dissolving the progestagenic compound in an organic solvent to form a solution;
(ii) mixing the resulting solution comprising the progestogenic compound with a carrier;
(iii) optionally granulating the resulting mixture;
(iv) drying the mixture;
(v) admixing the mixture with excipients; and
(vi) turning the resulting mixture into dosage units wherein the progestagenic compound is etonogestrel.

2. The method according to claim 1, wherein the mixture is filled into a capsule so as to make a dosage unit in the form of a capsule.

3. The method according to claim 1, wherein the mixture is tabletted so as to make a dosage unit in the form of a tablet.

4. (canceled)

5. A tablet comprising etonogestrel wherein after storage for 12 months at 25° C., at least 95% of the etonogestrel is still present in the tablet.

6. A tablet comprising etonogestrel wherein after storage for 12 months at 25° C., at least 96% of the etonogestrel is still present in the tablet.

7. A tablet comprising etonogestrel wherein after storage for 12 months at 25° C., at least 97% of the etonogestrel is still present in the tablet.

8. A tablet comprising etonogestrel wherein after storage for 12 months at 25° C., at least 98% of the etonogestrel is still present in the tablet.

9. A tablet comprising etonogestrel wherein after storage for 12 months at 25° C., at least 99% of the etonogestrel is still present in the tablet.
